(19) United States
(12) Patent Application Publication
(10) Pub. No.: US 2026/0273289 A1
(43) Pub. Date: Sep. 17, 2026

(54) IMPLANTABLE MEDICAL DEVICES WITH PHOTOCURABLE FEEDTHROUGH POTTING ADHESIVES AND METHODS OF MANUFACTURE

(71) Applicant: MEDTRONIC, INC., Minneapolis, MN (US)

(72) Inventors: Yangbin Chen, Lake Elmo, MN (US); Dena A. Laramy, St. Paul, MN (US)

(21) Appl. No.: 19/565,843

(22) Filed: Mar. 13, 2026

Related U.S. Application Data
(60) Provisional application No. 63/771,758, filed on Mar. 14, 2025.

(51) Int. Cl. A61N 1/375; C08G 59/22; C08G 59/24; C08G 59/40; C08G 59/62; C09J 5/00; C09J 163/00

(52) U.S. Cl. CPC A61N 1/3754; C08G 59/223; C08G 59/226; C08G 59/24; C08G 59/245; C08G 59/4007; C08G 59/62; C09J 5/00; C09J 163/00

(57) ABSTRACT

An implantable medical device with a photocurable liquid feedthrough potting adhesive and a feedthrough potting adhesive formed therefrom, and methods of making the same are provided. The feedthrough potting adhesive bonds a feedthrough pin within a feedthrough ferrule of the implantable medical device. The feedthrough potting adhesive includes a polyether polymer formed by reacting ingredients including a prepolymer compound, a cross-linking agent, a photoinitiator, and a hydrophobic polyol.

### RELATED APPLICATIONS

This application claims the benefit of U.S. Provisional Application Ser. No. 63/771,758, filed Mar. 14, 2025, the entire contents of which are incorporated herein by reference.

### TECHNICAL FIELD

This disclosure generally relates to, among other things, potting adhesives for use in implantable medical devices and, more specifically, to photocurable liquid feedthrough potting adhesives for use in feedthrough ferrules of implantable medical devices and medical devices including feedthrough ferrules with feedthrough potting adhesives formed therefrom.

### BACKGROUND

In general, there is a need for potting adhesive compositions that can provide fast cure on demand at relatively low temperatures, while also having a long pot life: maintaining consistent, low viscosity at room temperature (i.e., an ambient temperature of between 20° C. and 25° C. or an ambient temperature of approximately 23° C.). Such potting adhesive compositions may be useful, for example, for use in implantable medical devices such as implantable pulse generators, implantable cardioverter defibrillators, and neurostimulators.

Implantable medical devices generally use potting adhesive compositions to bond feedthrough pins within a feedthrough ferrule and to electrically insulate the pin from the ferrule. Potting adhesives may also commonly be used to isolate the internal components of the implantable medical device from the patient's body and the patient's bodily fluid. Because internal components (e.g., batteries, capacitors, processors, etc.) may commonly be sensitive to the aqueous environment of the patient's body, it is important to isolate sensitive internal components.

Important characteristics for feedthrough potting adhesive compositions may include, for example, viscosity, pot life, optical properties (e.g., transparency), mixing compatibility of the parts (such as the mixing compatibility of a resin and a hardener), cure time, and cure temperature. To isolate the internal components of an implantable medical device, biocompatibility, impedance, long-term stability, and durability of the feedthrough potting adhesive may also be important considerations, particularly for implantable medical devices intended for long-term use.

### SUMMARY

As described herein, photocurable liquid feedthrough potting adhesive compositions for implantable medical devices may be prepared with useful characteristics including low viscosity, free-flowing and self-leveling properties, and photoinitiated curing. Photoinitiated curing may be described as affording a long pot life that allows pre-mixing of the photocurable liquid feedthrough potting adhesive and cure on-demand. In some embodiments, the photocurable liquid feedthrough potting adhesive compositions may be described as having an indefinite pot life, particularly compared with, for example, thermally-curable liquid feedthrough potting adhesive compositions. In comparison, thermally-curable liquid feedthrough potting adhesives generally begin curing upon the combination of resin and hardener and, therefore, must be mixed shortly before use, and furthermore tend to have a relatively short pot life, within which the liquid adhesive must be used.

As further described herein, feedthrough potting adhesives formed by reacting ingredients of photocurable liquid feedthrough potting adhesive compositions may be prepared that advantageously have long-term stability afforded by characteristics including low water uptake, hydrolytic stability, resistance to oxidative degradation, and glass transition temperatures greater than human body temperature. Furthermore, feedthrough potting adhesives formed by reacting ingredients of photocurable liquid feedthrough may be prepared, as described herein, having useful characteristics such as high impedance (e.g., 1 gigaohm or greater at 0.1 Hz), low current leakage (e.g., close to zero milliAmps), and storage and loss modulus suitable for mechanical support of a feedthrough pin bonded within a feedthrough ferrule of a feedthrough assembly.

In one example, aspects of this disclosure relate to an implantable medical device feedthrough assembly with a feedthrough ferrule and a feedthrough potting adhesive in the feedthrough ferrule, the feedthrough potting adhesive having a polyether polymer formed by reacting ingredients including: a photoinitiator, a hydrophobic polyol, a cross-linking agent, and a prepolymer compound. The prepolymer compound includes segments of the formula X₁—R—X₂, wherein X₁ is an epoxide, a glycidyl ether, an oxetane, or a photoinitiated ring-opening cyclic ether, wherein X₂ is an epoxide, a glycidyl ether, an oxetane, a photoinitiated ring-opening cyclic ether, or a hydrogen, and wherein R is an organic group.

In another example, aspects of this disclosure relate to a method for forming an implantable medical device, the method including bonding a feedthrough pin within a ferrule with a feedthrough potting adhesive and curing the feedthrough potting adhesive. The feedthrough potting adhesive includes a polyether polymer formed by reacting ingredients including: a photoinitiator, a hydrophobic polyol, a cross-linking agent, and a prepolymer compound. The prepolymer compound includes segments of the formula X₁—R—X₂, wherein X₁ is an epoxide, a glycidyl ether, an oxetane, or a photoinitiated ring-opening cyclic ether, wherein X₂ is an epoxide, a glycidyl ether, an oxetane, a photoinitiated ring-opening cyclic ether, or a hydrogen, and wherein R is an organic group.

In yet another example, aspects of this disclosure relate to a feedthrough potting adhesive with a polyether polymer formed by reacting ingredients including: a photoinitiator, a hydrophobic polyol, a cross-linking agent, and a prepolymer compound. The prepolymer compound includes segments of the formula X₁—R—X₂, wherein X₁ is an epoxide, a glycidyl ether, an oxetane, or a photoinitiated ring-opening cyclic ether, wherein X₂ is an epoxide, a glycidyl ether, an oxetane, a photoinitiated ring-opening cyclic ether, or a hydrogen, and wherein R is an organic group.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is an illustrative implantable medical device implanted in a patient including a feedthrough potting adhesive formed by a photocurable feedthrough potting adhesive.

FIG. 2 is a cross-section side view of an illustrative feedthrough assembly including the feedthrough potting adhesive of the implantable medical device of FIG. 1.

FIG. 3 is a schematic of an illustrative method of forming the implantable medical device of FIG. 1 including the feedthrough potting adhesive in the feedthrough assembly of FIG. 2.

FIG. 4 is a graphical representation showing the pot time of curable liquid feedthrough potting adhesives.

FIG. 5 is a graphical representation showing the impedance of feedthrough potting adhesives.

The figures are rendered primarily for clarity and, as a result, are not necessarily drawn to scale. Moreover, various structure/components may be shown diagrammatically or removed from some of or all the views to better illustrate aspects of the depicted embodiments, or where inclusion of such structure/components is not necessary to an understanding of the various exemplary embodiments described herein. The lack of illustration/description of such structures/components in a particular figure is, however, not to be interpreted as limiting the scope of the various embodiments in any way.

### DETAILED DESCRIPTION

All scientific and technical terms used herein have meanings commonly used in the art unless, otherwise specified. The definitions provided herein are to facilitate understanding of certain terms used frequently herein and are not meant to limit the scope of the present disclosure.

Unless otherwise indicated, the terms “polymer”, “polymerized monomers”, and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The term “substantially” as used here has the same meaning as “significantly,” and can be understood to modify the term that follows by at least 90%, at least 95%, or at least 98%, unless the content clearly dictates otherwise. The term “substantially free” of a particular compound means that the compositions of the present invention contain less than 1,000 parts per million (ppm) of the recited compound. The term “essentially free” of a particular compound means that the compositions of the present invention contain less than 100 parts per million (ppm) of the recited compound. The term “completely free” of a particular compound means that the compositions of the present invention contain less than 20 parts per billion (ppb) of the recited compound. In the context of the aforementioned phrases, the compositions of the present invention contain less than the aforementioned amount of the compound whether the compound itself is present in unreacted form or has been reacted with one or more other materials.

In this disclosure, all numbers are assumed to be modified by the term “about,” which encompasses the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to”, “at most”, or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure. Such inclusive or open-ended words encompass more restrictive or closed terms or phrases, such as “consisting” or “consisting essentially.”

As used herein, “consisting essentially of” means that the article or method consisting essentially of listed elements may include additional elements that do not materially affect the basic and novel characteristics of the article or method.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Reference in the specification to “some embodiments,” “an embodiment,” “one embodiment,” “embodiments,” “one or more embodiments,” “at least one embodiment,” or “other embodiments” means that a particular feature, structure, or characteristic described in connection with the embodiments is included in at least some embodiments, but not necessarily all embodiments, of the present disclosures.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components.

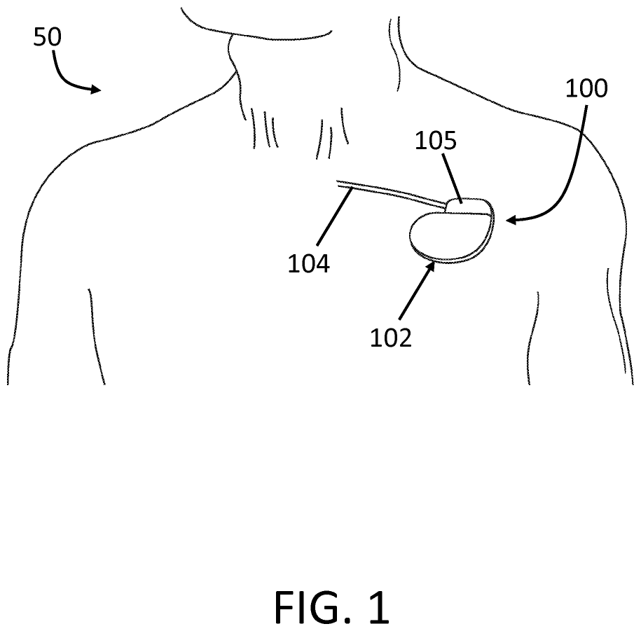

As described herein, an implantable medical device may be provided including a feedthrough potting adhesive composition formed from a photocurable feedthrough potting adhesive. An illustrative implantable medical device 100 implanted in a patient 50 including the feedthrough potting adhesive is shown in FIG. 1. The implantable medical device 100 may include a case 102, which may also be referred to as a housing. The case 102 may house one or more internal components of the implantable medical device 100, such as one or more batteries, electrical circuits, and the like. The implantable medical device 100 may further include one or more external components, such as a lead 104. The one or more external components may be electrically connectable to the one or more internal components via a feedthrough, which may be positioned in a header 105. The header 105 may form a unitary part with, or may be coupled to, the case 102.

The header 105 may define a bore through which a portion of the lead 104 may be inserted. The lead 104 may have electrical contacts that electrically couple with contacts in the header 105 when the lead 104 is inserted into the bore. The electrical contacts in the header 105 may be electrically coupled to the electrical components of the implantable medical device 100 through one or more feedthroughs.

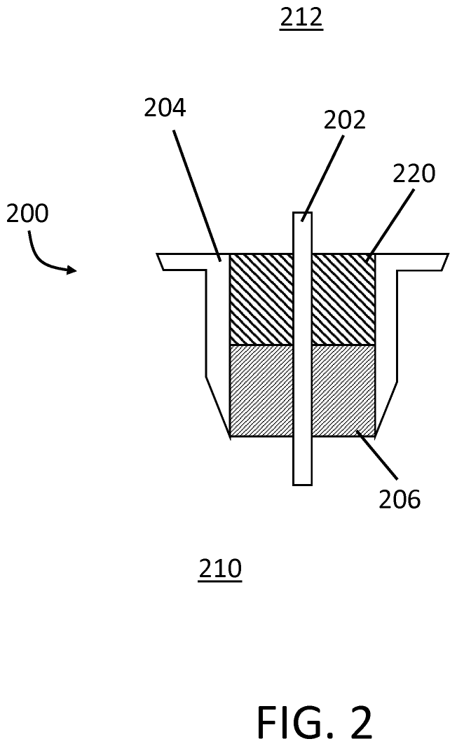

An illustrative feedthrough assembly 200 including the feedthrough potting adhesive 220 is shown in FIG. 2. The feedthrough assembly 200 may include a feedthrough pin 202 extending and providing electrical connection between an internal volume 210 (e.g., within the case 102 shown in FIG. 1) of an implantable medical device (e.g., the implantable medical device 100) and an outside environment 212 (e.g., within the header 105 shown in FIG. 1, which may define a bore in communication with a patient's bodily fluid). The feedthrough assembly 200 may include a ferrule 204 having at least a portion disposed between the internal volume 210 and the outside environment 212. The ferrule 204 may be tubular. The feedthrough pin 202 may extend through the ferrule 204.

The feedthrough assembly 200 may include a plug element 206 disposed at least partially within the ferrule 204. The feedthrough pin 202 may extend through a lumen in the plug element 206.

In one or more embodiments, the feedthrough assembly 200 includes the feedthrough potting adhesive 220. The adhesive 220 may be disposed in the ferrule 204. The pin 202 may extend through the adhesive 220. The adhesive 220 may provide a bond between the pin 202 and the ferrule 204. The adhesive 220, as described herein, may provide a seal between the outside environment 212 and the internal volume 210. The adhesive 220 may additionally or alternatively provide electrical insulation between the ferrule 204 and the pin 202.

In at least one embodiment of the feedthrough potting adhesive composition includes a polyether polymer formed by reacting ingredients including a prepolymer composition, a photoinitiator, a cross-linking agent, and a hydrophobic polyol. The composition of the feedthrough potting adhesive may be affected by the selection and amounts of the reaction ingredients. The composition of the feedthrough potting adhesive may be selected for characteristics such as impedance, dielectric constant, loss modulus, storage modulus, and water uptake, as examples. As another example, the composition of the feedthrough potting adhesive may be selected for optical properties (e.g., optical clarity, transparency, etc.). In some embodiments, the composition of the feedthrough potting adhesive is selected to afford a feedthrough potting adhesive that is at least substantially transparent (e.g., substantially transparent, essentially transparent, completely transparent, etc.). As used herein, the term “transparent” refers to a material's transmission of electromagnetic radiation incident on the material. Further, the phrases “substantially transparent,” “essentially transparent,” and “completely transparent” mean that the material transmits, respectively, greater than 50% greater than 90%, or greater than 99%, of electromagnetic radiation incident on the material for a selected wavelength or range of wavelengths, assuming no reflection at the air-material boundaries. In one or more embodiments, the feedthrough potting adhesive is substantially transparent to (i.e., transmissive to greater than 50% of), essentially transparent to (i.e., transmissive to greater than 90% of), or completely transparent to (i.e., transmissive to greater than 99% of) UV light, visible light, IR light, or any combination thereof. Transparency of the feedthrough potting adhesive may be advantageous, for example, to allow evaluation of the quality of adhesion between the cured feedthrough potting adhesive and the feedthrough assembly (e.g., adhesion between the cured feedthrough potting adhesive and the ferrule, adhesion between the cured feedthrough potting adhesive and the pin, etc.), such as evaluation based on the formation of gaps at the interface between the feedthrough potting adhesive and the feedthrough assembly.

The reaction ingredients that react to form the polyether polymer may be referred to herein as a “photocurable liquid feedthrough potting adhesive.” In other words, the photocurable liquid feedthrough potting adhesive may include the prepolymer composition, the photoinitiator, the cross-linking agent, and the hydrophobic polyol, and reaction ingredients of the photocurable liquid feedthrough potting adhesive may react to form the polyether polymer of the feedthrough potting adhesive. The composition of the photocurable liquid feedthrough potting adhesive may be selected for characteristics such as low viscosity (e.g., at room temperature), long pot life, fast curing at low temperature (i.e., when selectively exposed to ultraviolet (UV) radiation), and thixotropic index, as examples.

In one or more embodiments according to the present disclosure, the photocurable liquid feedthrough potting adhesive includes a prepolymer composition including a prepolymer compound (e.g., at least one prepolymer compound, one or more prepolymer compounds, two or more prepolymer compounds, three or more prepolymer compounds, four or more prepolymer compounds, etc.). Further, the polyether polymer of the feedthrough potting adhesive may be formed by reacting ingredients including a prepolymer composition including a prepolymer compound (e.g., at least one prepolymer compound, one or more prepolymer compounds, two or more prepolymer compounds, three or more prepolymer compounds, four or more prepolymer compounds, etc.). Prepolymer compounds may be described as starting materials (e.g., monomers, oligomers, precursor polymers, etc.) used to make a polymeric material (e.g., polyether polymer of the feedthrough potting adhesive).

The photocurable liquid feedthrough potting adhesive may include any suitable amount of prepolymer composition or prepolymer compounds thereof. Suitable amounts of prepolymer composition may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), curing conditions (e.g., UV light intensity, UV light wavelength, etc.), curing time, or optical properties (e.g., optical clarity, transparency, etc.), as examples. The photocurable liquid feedthrough potting adhesive may include 10 wt-% to 80 wt-% or 30 wt-% to 70 wt-% of prepolymer composition based on the total weight of the photocurable liquid feedthrough potting adhesive, for example. As further examples, the photocurable liquid feedthrough potting adhesive may include 10 wt-% or greater, 15 wt-% or greater, 20 wt-% or greater, 25 wt-% or greater, 30 wt-% or greater, 35 wt-% or greater, 40 wt-% or greater, 45 wt-% or greater, 50 wt-% or greater, 55 wt-% or greater, 60 wt-% or greater, 65 wt-% or greater, 70 wt-% or greater, 75 wt-% or greater, or 80 wt-% or greater, and/or 85 wt-% or less, 80 wt-% or less, 75 wt-% or less, 70 wt-% or less, 65 wt-% or less, 60 wt-% or less, 55 wt-% or less, 50 wt-% or less, 45 wt-% or less, 40 wt-% or less, 35 wt-% or less, 30 wt-% or less, 25 wt-% or less, 20 wt-% or less, 15 wt-% or less, or 10 wt-% or less of the prepolymer composition based on the total weight of the photocurable liquid feedthrough potting adhesive.

The photocurable liquid feedthrough potting adhesive may include any suitable prepolymer composition, or any suitable combination of prepolymer compounds thereof. Suitable prepolymer compounds may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), non-polarity, octanol-water partition coefficient (Log P), material (e.g., mixing) compatibility with other ingredients of the photocurable liquid feedthrough potting adhesive (e.g., with the photoinitiator, the hydrophobic polyol, the cross-linker, etc.), material compatibility with the implantable medical device (e.g., with the ferrule material, the pin material, etc.), or optical properties (e.g., optical clarity, transparency, etc.), as examples. Suitable prepolymer compounds may include, for example, photoinitiated prepolymer compounds, such as prepolymer compounds including photoinitiated ring-opening cyclic ether groups. A photoinitiated ring-opening cyclic ether may be described as an organic compound with a ring structure containing an oxygen atom. In some embodiments, suitable prepolymer compounds include segments of Formula I or may be derived from segments of Formula I:

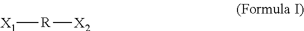

In Formula I, each of X₁ and X₂ may independently be a cyclic ether. In one or more embodiments, each of X₁ and X₂ of Formula I is individually a photoinitiated ring-opening cyclic ether, an epoxide, an oxetane, or a glycidyl ether. In some embodiments, X₂ of Formula I is a Hydrogen. In Formula I, R may be an organic group. In one or more embodiments, R of Formula I is an aliphatic group, an alkyl group, an alkyl ether group, a cycloalkane group, an aryl group, or a cycloaliphatic organic group.

As another example, suitable prepolymer compounds may include a diepoxide compound or may be derived from a diepoxide compound, such as the diepoxide compound of Formula II:

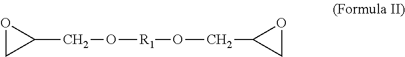

In Formula II, R₁ is an organic group, an alkyl group, an alkyl ether group, a cycloalkane group, a methyl group, an ethyl group, a propyl group, a butyl group, a pentyl group, a hexyl group, a heptyl group, an octyl group, a nonyl group, a decyl group, an undecyl group, a dodecyl group, a tridecyl group, a tetradecyl group, 2,2-bis(cyclohexyl)propane, dimethyl cyclohexane, dimethyl tricyclodecane, or an aryl group. Suitable prepolymer compounds may include segments of Formula I where X₁, X₂, or both are glycidyl ethers, and where R is O—R₁—O, as further examples. As still further examples, suitable prepolymer compounds may include diglycidyl ether of bisphenol A resin (“DGEBPA”, available, e.g., from WESTLAKE EPOXY INC. of Stafford, Texas, under the trade name EPON RESIN 828), butyl glycidyl ether resin (“BGE”, available, e.g., from WESTLAKE EPOXY INC. of Stafford, Texas, under the trade name HELOXY MODIFIER 61), 1,4-butanediol diglycidyl ether (“BDDGE”, available, e.g., from WESTLAKE EPOXY INC. of Stafford, Texas, under the trade name HELOXY MODIFIER 67), tricyclodecane dimethanol diglycidylether (“TCDDGE”, available, e.g., from ADEKA CORPORATION of Tokyo, Japan, under the trade name EP4088S), hydrogenated bisphenol A type epoxy resin (“HBPADGE”, available, e.g., from ADEKA CORPORATION of Tokyo, Japan, under the trade name EP-4080E), propoxylated diglycidyl ether of bisphenol A resin (“DGEBPA-PO”, available, e.g., from ADEKA CORPORATION of Tokyo, Japan, under the trade name EP-4000S), or Glycidyl 3-(trimethoxysilyl)propyl ether (“GLYMO”, available, e.g., from EVONIK CORPORATION of Parsippany, New Jersey, under the trade name DYNASYLAN GLYMO).

In embodiments where the photocurable liquid feedthrough potting adhesive includes a prepolymer composition including a diepoxide compound, the photocurable liquid feedthrough potting adhesive may include any suitable amount of the diepoxide compound. Suitable amounts of the diepoxide compound may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), curing conditions (e.g., UV light intensity, UV light wavelength, etc.), curing time, or optical properties (e.g., optical clarity, transparency, etc.), as examples. The photocurable liquid feedthrough potting adhesive may include 30 wt-% to 60 wt-% or 40 wt-% to 50 wt-% of the diepoxide compound based on the total weight of the photocurable liquid feedthrough potting adhesive, for example. As further examples, the photocurable liquid feedthrough potting adhesive may include 25 wt-% or greater, 30 wt-% or greater, 35 wt-% or greater, 40 wt-% or greater, 45 wt-% or greater, 50 wt-% or greater, 55 wt-% or greater, or 60 wt-% or greater, and/or 65 wt-% or less, 60 wt-% or less, 55 wt-% or less, 50 wt-% or less, 45 wt-% or less, 40 wt-% or less, 35 wt-% or less, or 30 wt-% or less of the diepoxide compound based on the total weight of the photocurable liquid feedthrough potting adhesive.

As yet another example, suitable prepolymer compounds may include a dioxetane compound or may be derived from a dioxetane compound, such as the dioxetane compound of Formula III:

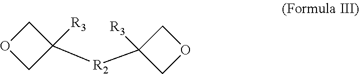

In Formula III, R₂ is an organic group, an alkyl group, an alkyl ether group, a cycloalkane group, a methyl group, an ethyl group, a propyl group, a butyl group, a pentyl group, a hexyl group, a heptyl group, an octyl group, a nonyl group, a decyl group, an undecyl group, a dodecyl group, a tridecyl group, a tetradecyl group; dimethyl ether, diethyl ether, dipropyl ether, or an aryl group. Suitable prepolymer compounds may include segments of Formula I where X₁, X₂, or both are oxetanes, and where R is R₂, as further examples. As still further examples, suitable prepolymer compounds may include 3,3′-(oxybis(methylene))bis(3-ethyloxetane) (“DOX”, available, e.g., from PERSTORP SPECIALTY CHEMICALS AB of Perstorp, Sweden, under the trade name CURALITE OXPLUS) or 3-ethyl-3-hydroxymetyloxetane (“EOXA”, available, e.g., from PERSTORP SPECIALTY CHEMICALS AB of Perstorp, Sweden, under the trade name CURALITE OX).

In embodiments where the photocurable liquid feedthrough potting adhesive includes prepolymer compositions including a dioxetane compound, the photocurable liquid feedthrough potting adhesive may include any suitable amount of the dioxetane compound. Suitable amounts of the dioxetane compound may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), curing conditions (e.g., UV light intensity, UV light wavelength, etc.), curing time, or optical properties (e.g., optical clarity, transparency, etc.), as examples. The photocurable liquid feedthrough potting adhesive may include 10 wt-% to 50 wt-% or 25 wt-% to 45 wt-% of the dioxetane compound based on the total weight of the photocurable liquid feedthrough potting adhesive, for example. As further examples, the photocurable liquid feedthrough potting adhesive may include 5 wt-% or greater, 10 wt-% or greater, 15 wt-% or greater, 20 wt-% or greater, 25 wt-% or greater, 30 wt-% or greater, 35 wt-% or greater, 40 wt-% or greater, 45 wt-% or greater, or 50 wt-% or greater, and/or 55 wt-% or less, 50 wt-% or less, 45 wt-% or less, 40 wt-% or less, 35 wt-% or less, 30 wt-% or less, 25 wt-% or less, 20 wt-% or less, 15 wt-% or less, or 10 wt-% or less of the dioxetane compound based on the total weight of the photocurable liquid feedthrough potting adhesive.

In some embodiments, as described herein, the photocurable liquid feedthrough potting adhesive includes two or more prepolymer compounds. For example, the photocurable liquid feedthrough potting adhesive may include one or more (e.g., two, three, four, or more) diepoxide compounds (e.g., according to Formula II) and one or more (e.g., two, three, four, or more) dioxetane compounds (e.g., according to Formula III). In one or more embodiments, the photocurable liquid feedthrough potting adhesive includes 45 wt-% of a diepoxide (e.g., HBPADGE) and 27 wt-% of a dioxetane (e.g., DOX) based on the total weight of the photocurable liquid feedthrough potting adhesive. In at least one embodiment, the photocurable liquid feedthrough potting adhesive includes 45 wt-% of a diepoxide (e.g., TCDDGE) and 30 wt-% of a dioxetane (e.g., DOX) based on the total weight of the photocurable liquid feedthrough potting adhesive.

In one or more embodiments, suitable prepolymer compound may be non-polar, substantially non-polar, hydrophobic, or substantially hydrophobic. Suitable prepolymer compositions or prepolymer compounds thereof may have any suitable octanol-water partition coefficient (Log P). Suitable prepolymer compound octanol-water partition coefficients may include, for example, 2 to 15 or 3 to 10. As further examples, suitable prepolymer compound octanol-water partition coefficients may include 1.5 or greater, 2 or greater, 3 or greater, 5 or greater, or 10 or greater. Octanol-water partition coefficient may be determined according to ASTM E1147-92 (1997), “Standard Test Method for Partition Coefficient (N-Octanol/Water) Estimation by Liquid Chromatography.”

In one or more embodiments according to the present disclosure, the photocurable liquid feedthrough potting adhesive includes a photoinitiator (e.g., at least one photoinitiator). Further, the polyether polymer of the feedthrough potting adhesive may be formed by reacting ingredients including a photoinitiator (e.g., at least one photoinitiator). Photoinitiators may be described as materials that release reactive species (e.g., free radicals, cations, etc.) as a result of absorbing electromagnetic radiation (e.g., UV light). The photocurable liquid feedthrough potting adhesive may include any suitable photoinitiator, or any suitable combination of suitable photoinitiators. Suitable photoinitiators may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), non-polarity, octanol-water partition coefficient (Log P), material (e.g., mixing) compatibility with other ingredients of the photocurable liquid feedthrough potting adhesive (e.g., with the prepolymer composition, the hydrophobic polyol, the cross-linking agent, etc.), or material compatibility with the implantable medical device (e.g., with the ferrule material, the pin material, etc.), as examples. In some embodiments, suitable photoinitiators are selected based on light absorption profiles. For example, suitable photoinitiators may have light absorption profiles including wavelengths of 200 nm to 450 nm. Suitable photoinitiators may include, for example, a cation from an aromatic diazonium salt, an aromatic sulfonium salt, an aromatic iodonium salt, a metallocene-based compound, an aromatic phosphonium salt, or any combination thereof, and an anion from BF₄⁻, AsF₆⁻, SbF₆⁻, PF₆⁻, [B(CF₃)₄]⁻, B(C₆F₅)₄⁻, B[C₆H₃-3,5(CF₃)₂]₄⁻, B(C₆H₄CF₃)₄⁻, B(C₆H₃F₂)₄⁻, B[C₆F₄-4(CF₃)]₄⁻, or any combination thereof. In one embodiment, the photoinitiator is, or includes, a cation from a triarylsulfonium salt and an anion from hexafluoroantimonate.

The photocurable liquid feedthrough potting adhesive may include any suitable amount of photoinitiator or photoinitiators (e.g., one or more photoinitiators, two or more photoinitiators, three or more photoinitiators, four or more photoinitiators, etc.). Suitable photoinitiator amounts may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), curing time, or light absorption profiles, as examples. The photocurable liquid feedthrough potting adhesive may include 0.1 wt-% to 5 wt-% of photoinitiator based on the total weight of the photocurable liquid feedthrough potting adhesive, for example. As further examples, the photocurable liquid feedthrough potting adhesive may include 0.1 wt-% or greater, 0.5 wt-% or greater, 1 wt-% or greater, 2 wt-% or greater, 3 wt-% or greater, 4 wt-% or greater, or 5 wt-% or greater, and/or 6 wt-% or less, 5 wt-% or less, 4 wt-% or less, 3 wt-% or less, 2 wt-% or less, 1 wt-% or less, 0.5 wt-% or less, or 0.1 wt-% or less of photoinitiator based on the total weight of the photocurable liquid feedthrough potting adhesive. It will be understood in view of the present disclosure that any suitable amount of photoinitiator may be used, and the disclosure is not limited in this regard. It will be further understood in view of the present disclosure that suitable amounts of photoinitiator may be selected based on factors such as those disclosed herein.

In one or more embodiments, suitable photoinitiators may be non-polar, substantially non-polar, hydrophobic, or substantially hydrophobic. Suitable photoinitiators may have any suitable octanol-water partition coefficient (Log P). Suitable photoinitiator octanol-water partition coefficients may include, for example, 2 to 15 or 3 to 10. As further examples, suitable photoinitiator octanol-water partition coefficients may include 1.5 or greater, 2 or greater, 3 or greater, 5 or greater, or 10 or greater.

In some embodiments according to the present disclosure, the photocurable liquid feedthrough potting adhesive includes a cross-linking agent (e.g., at least one cross-linking agent). Further, the polyether polymer of the feedthrough potting adhesive may be formed by reacting ingredients including a cross-linking agent (e.g., at least one cross-linking agent). The photocurable liquid feedthrough potting adhesive may include any suitable cross-linking agent, or any suitable combination of suitable cross-linking agents. Suitable cross-linking agents may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), non-polarity, octanol-water partition coefficient (Log P), material (e.g., mixing) compatibility with other ingredients of the photocurable liquid feedthrough potting adhesive (e.g., with the prepolymer composition, the photoinitiator, the hydrophobic polyol, etc.), material compatibility with the implantable medical device (e.g., with the ferrule material, the pin material, etc.), or optical properties (e.g., optical clarity, transparency, etc.), as examples. Suitable cross-linking agents may include, for example, glycidyl ethers of alkoxy silanes, glycidyl ethers of silsesquioxane, glycidyl 3-(trimethoxysilyl)propyl ether, or any combination thereof.

The photocurable liquid feedthrough potting adhesive may include any suitable amount of cross-linking agent or cross-linking agents (e.g., two cross-linking agents, three cross-linking agents, four cross-linking agents, or more). Suitable cross-linking agent amounts may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), curing time, or optical properties (e.g., optical clarity, transparency, etc.), as examples. The photocurable liquid feedthrough potting adhesive may include 0.5 wt-% to 5 wt-% of cross-linking agent based on the total weight of the photocurable liquid feedthrough potting adhesive, for example. As further examples, the photocurable liquid feedthrough potting adhesive may include 0.3 wt-% or greater, 0.5 wt-% or greater, 1 wt-% or greater, 2 wt-% or greater, 3 wt-% or greater, 4 wt-% or greater, or 5 wt-% or greater, and/or 6 wt-% or less, 5 wt-% or less, 4 wt-% or less, 3 wt-% or less, 2 wt-% or less, 1 wt-% or less, or 0.5 wt-% or less of cross-linking agent based on the total weight of the photocurable liquid feedthrough potting adhesive. It will be understood in view of the present disclosure that any suitable amount of cross-linking agent may be used, and the disclosure is not limited in this regard. It will be further understood in view of the present disclosure that suitable amounts of cross-linking agent may be selected based on factors such as those disclosed herein.

In one or more embodiments, suitable cross-linking agents may be non-polar, substantially non-polar, hydrophobic, or substantially hydrophobic. Suitable cross-linking agents may have any suitable octanol-water partition coefficient (Log P). Suitable cross-linking agent octanol-water partition coefficients may include, for example, 2 to 15 or 3 to 10. As further examples, suitable cross-linking agent octanol-water partition coefficients may include 1.5 or greater, 2 or greater, 3 or greater, 5 or greater, or 10 or greater.

In at least one embodiment according to the present disclosure, the photocurable liquid feedthrough potting adhesive includes a hydrophobic polyol (e.g., at least one hydrophobic polyol). Further, the polyether polymer of the feedthrough potting adhesive may be formed by reacting ingredients including a hydrophobic polyol (e.g., at least one hydrophobic polyol). Hydrophobic polyols may be described as a polyol with an octanol-water-partition coefficient (Log P) of 0.5 or greater. In some embodiments, a hydrophobic polyol is described as a polyol with a Log P of 1 or greater, 1.5 or greater, 2 or greater, 3 or greater, 5 or greater, or 8 or greater. The photocurable liquid feedthrough potting adhesive may include any suitable hydrophobic polyol, or any suitable combination of suitable hydrophobic polyols. Suitable hydrophobic polyols may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), non-polarity, octanol-water partition coefficient (Log P), material (e.g., mixing) compatibility with other ingredients of the photocurable liquid feedthrough potting adhesive (e.g., with the prepolymer, the photoinitiator, the cross-linker, etc.), material compatibility with the implantable medical device (e.g., with the ferrule material, the pin material, etc.), or optical properties (e.g., optical clarity, transparency, etc.), as examples. Suitable hydrophobic polyols may include, for example, a polyether polyol, a polyester polyol, a dimer-diol, a trimer-triol, a hydroxyl-terminated hydrogenated polybutadiene, a hydrogenated hydroxyl-terminated polyfarnesene, or any combination thereof. In one or more embodiments, the hydrophobic polyol is dimer diol (available, e.g., from CRODA INC., of Plainsboro, NJ, under the trade name PRIPOL 2043).

The photocurable liquid feedthrough potting adhesive may include any suitable amount of hydrophobic polyol or hydrophobic polyols (e.g., two or more hydrophobic polyols, three or more hydrophobic polyols, four or more hydrophobic polyols, etc.). Suitable hydrophobic polyols amounts may be selected based on factors such as desired characteristics of the photocurable liquid feedthrough potting adhesive (e.g., viscosity, thixotropic index, octanol-water-partition coefficient, etc.), desired characteristics of the feedthrough potting adhesive (e.g., loss modulus, storage modulus, impedance, dielectric constant, etc.), curing time, or optical properties (e.g., optical clarity, transparency, etc.), as examples. The photocurable liquid feedthrough potting adhesive may include 5 wt-% to 35 wt-% of hydrophobic polyol based on the total weight of the photocurable liquid feedthrough potting adhesive, for example. As further examples, the photocurable liquid feedthrough potting adhesive may include 3 wt-% or greater, 5 wt-% or greater, 10 wt-% or greater, 15 wt-% or greater, 20 wt-% or greater, 25 wt-% or greater, 30 wt-% or greater, or 35 wt-% or greater and/or 40 wt-% or less, 35 wt-% or less, 30 wt-% or less, 25 wt-% or less, 20 wt-% or less, 15 wt-% or less, 10 wt-% or less, or 5 wt-% or less of hydrophobic polyol based on the total weight of the photocurable liquid feedthrough potting adhesive. It will be understood in view of the present disclosure that any suitable amount of hydrophobic polyol may be used, and the disclosure is not limited in this regard. It will be further understood in view of the present disclosure that suitable amounts of hydrophobic polyol may be selected based on factors such as those disclosed herein.

In one or more embodiments, suitable hydrophobic polyols may be non-polar, substantially non-polar, hydrophobic, or substantially hydrophobic. Suitable hydrophobic polyols may have any suitable octanol-water partition coefficient (Log P). Suitable hydrophobic polyol octanol-water partition coefficients may be, or include, for example, 5 to 20 or 10 to 15. As further examples, suitable hydrophobic polyol Log P values may be, or include, 4 or greater, 5 or greater, 6 or greater, 7 or greater, 8 or greater, 10 or greater, 12 or greater, 15 or greater, 17 or greater, or 20 or greater, and/or 25 or less, 20 or less, 17 or less, 15 or less, 12 or less, 10 or less, 8 or less, 7 or less, 6 or less, or 5 or less.

The photocurable liquid feedthrough potting adhesive may have any suitable viscosity at room temperature. Further, the combination of ingredients reacted to form the polyether polymer of the feedthrough potting adhesive may have any suitable viscosity at room temperature. Viscosity may be determined (e.g., measured) using any suitable technique. Suitable techniques for determining viscosity may include, for example, ASTM D1084. Suitable viscosities at room temperature may be selected based on the flow and wetting characteristics desirable for effective flow and wetting of a feedthrough ferrule (e.g., the ferrule 204), for example. As another example, suitable viscosities at room temperature may be selected based on capability of the photocurable liquid feedthrough potting adhesive to be dispensed through a needle. Suitable viscosities at room temperature may be, or include, for example, 100 centipoise (cps) to 2500 cps or 200 cps to 1000 cps. As further examples, suitable viscosities at room temperature may be, or include, 50 cps or greater, 100 cps or greater, 150 cps or greater, 200 cps or greater, 300 cps or greater, 500 cps or greater, 700 cps or greater, 1000 cps or greater, 1300 cps or greater, 1500 cps or greater, 1700 cps or greater, 2000 cps or greater, or 2500 cps or greater, and/or 3000 cps or less, 2500 cps or less, 2000 cps or less, 1700 cps or less, 1500 cps or less, 1300 cps or less, 1000 cps or less, 700 cps or less, 500 cps or less, 300 cps or less, 200 cps or less, or 100 cps or less.

In one or more embodiments, the combination of ingredients that reacts to from the polyether polymer (i.e., the reaction ingredients of the photocurable liquid feedthrough potting adhesive) advantageously maintains a suitable viscosity (e.g., 200 cps to 2500 cps, suitable to be dispensed through a needle, etc.) for a long period of time (e.g., 2 hours or more) after combining the reaction ingredients and before curing (e.g., the curing 304 of the method 300 described herein). In other words, the combination of ingredients (i.e., the photocurable liquid feedthrough potting adhesive) may have a long pot life. The combination of ingredients (i.e., the photocurable liquid feedthrough potting adhesive) may have any suitable pot life. In other words, the combination of ingredients may have a suitable viscosity (e.g., less than 2500 cps), as described herein, for any suitable amount of time. Suitable pot lives (e.g., at an ambient temperature of 23° C., or room temperature) may include, for example, 1 hour or greater, 2 hours or greater, 3 hours or greater, 4 hours or greater, 5 hours or greater, 10 hours or greater, 24 hours or greater, 35 hours or greater, 50 hours or greater, 75 hours or greater, 100 hours or greater, or 120 hours or greater.

The photocurable liquid feedthrough potting adhesive may have any suitable thixotropic index. Further, the combination of ingredients reacted to form the polyether polymer of the feedthrough potting adhesive may have any suitable thixotropic index. Thixotropic index may be defined as the ratio of the viscosity (e.g., at room temperature) at 1/s shear rate over the viscosity at 10/s shear rate. Suitable thixotropic indexes may be selected based on factors such as flow and wetting characteristics desirable for effective flow and wetting of a feedthrough ferrule (e.g., the ferrule 204), for example. A low thixotropic index of 1.0 to 1.2 may be desirable for feedthrough potting applications, for example, because a low thixotropic index of 1.0 to 1.2 may be described as indicative of a liquid with free flowing and self-leveling properties. The photocurable liquid feedthrough potting adhesive may have a thixotropic index of 1.0 to 1.2. In certain embodiments, the photocurable feedthrough potting adhesive has a thixotropic index of 1.0.

In one or more embodiments, the photocurable liquid feedthrough potting adhesive may be non-polar, substantially non-polar, hydrophobic, or substantially hydrophobic. As described herein, non-polarity and/or hydrophobicity of the photocurable liquid feedthrough potting adhesive and the feedthrough potting adhesive (i.e., the cured photocurable liquid feedthrough potting adhesive) may be desirable, for example, to reduce or minimize water uptake (e.g., water uptake of the feedthrough potting adhesive). Low water uptake may be affected by or based on characteristics such as hydrophobicity and non-polarity of compounds in the photocurable liquid feedthrough potting adhesive and compounds in the feedthrough potting adhesive, as just two examples. For example, the feedthrough potting adhesive may be substantially non-polar. That is to say, the compounds included in the feedthrough potting adhesive may be non-polar or substantially non-polar. In such embodiments, the reaction ingredients reacted to form the feedthrough potting adhesive may be non-polar or substantially non-polar.

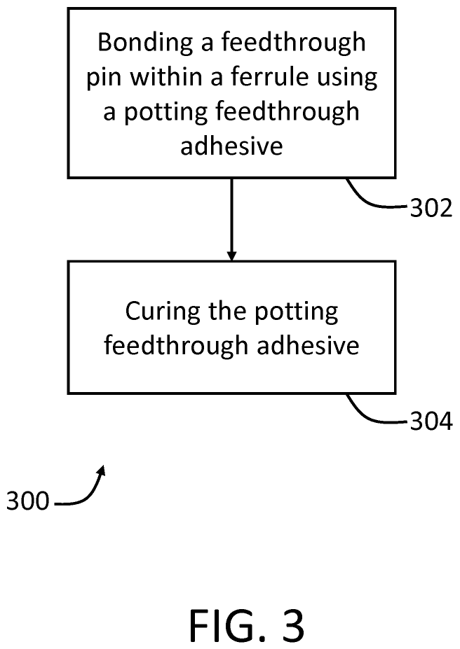

An illustrative method 300 for forming an implantable medical device (e.g., the implantable medical device 100) including the feedthrough potting adhesive (e.g., using the liquid curable feedthrough potting adhesive) is shown in FIG. 3. In one or more embodiments, the method 300 includes bonding 302 a feedthrough pin (e.g., the pin 202) of the implantable medical device within a ferrule (e.g., the ferrule 204) of the implantable medical device. The bonding 302 may be done using a photocurable liquid feedthrough potting adhesive according to one or more embodiments described herein. Further, the bonding 302 may be done using the combination of ingredients reacted to form the polyether polymer of the feedthrough potting adhesive according to one or more embodiments described herein. The bonding 302 may include applying (e.g., depositing) a combination of ingredients (e.g., a photocurable liquid feedthrough potting adhesive according to one or more embodiments described herein) that react form the polyether polymer of the feedthrough potting adhesive into the feedthrough ferrule, for example, through a needle.

In some embodiments, the method 300 includes curing 304 the photocurable liquid feedthrough potting adhesive to thereby form the cured feedthrough potting adhesive (e.g., within the ferrule). The curing 304 may occur for a cure time. For example, the curing 304 may include exposing the combination of ingredients that react form the polyether polymer of the feedthrough potting adhesive to UV (e.g., UV light, UV radiation, etc.) for a cure time.

The curing 304 may include any suitable cure time. Further, the feedthrough potting adhesive may be formed after any suitable cure time. Similarly, the polyether polymer of the feedthrough potting adhesive may be formed after any suitable cure time. Cure time may be described as the time under curing conditions (e.g., exposed to UV light of a designated wavelength, intensity, etc.) after which the photocurable liquid feedthrough potting adhesive reaches 99% cure. Degree of cure may be determined (e.g., measured) using any suitable technique. Suitable techniques for determining degree of cure include, for example, ASTM D3418 using differential scanning calorimetry (DSC). Suitable cure times may be affected by factors such as characteristics of the UV (e.g., wavelength, intensity, etc.), and characteristics of the reaction materials (e.g., characteristics of the prepolymer composition, the photoinitiator, the hydrophobic polyol, the cross-linking agent, etc.), as examples. Generally, a shorter cure time (e.g., 1 minute or less) is more desirable than a longer cure time. Suitable cure times may include, for example, 5 seconds to 1 minute or 5 seconds to 30 minutes. As further examples, suitable cure times may be, or include, 10 seconds or greater, 15 seconds or greater, 30 seconds or greater, 60 seconds or greater, 90 seconds or greater, 2 minutes or greater, 5 minutes or greater, 10 minutes or greater, 15 minutes or greater, 20 minutes or greater, or 30 minutes or greater, and/or 60 minutes or less, 30 minutes or less, 20 minutes or less, 15 minutes or less, 10 minutes or less, 5 minutes or less, 2 minutes or less, 90 seconds or less, 60 seconds or less, 30 seconds or less, or 15 seconds or less. It will be understood in view of this disclosure that any suitable cure times may be used, and the disclosure is not limited in this regard. It will further be understood in view of this disclosure that suitable cure times may be selected and affected based on factors such as those described herein.

The curing 304 may include using any suitable UV source, such as an LED lamp. Suitable UV sources may be selected based on factors such as wavelength and intensity of the UV provided. Suitable UV wavelengths may be, or include, between 100 nanometers (nm) and 405 nm, for example. As further examples, suitable UV wavelengths may be, or include, 100 nm or greater, 150 nm or greater, 200 nm or greater, 250 nm or greater, 300 nm or greater, 350 nm or greater, or 400 nm or greater, and/or 450 nm or less, 410 nm or less, 400 nm or less, 350 nm or less, 300 nm or less, 250 nm or less, 200 nm or less, or 150 nm or less. In certain embodiments, the UV wavelength may be 365 nm, 395 nm, or 405 nm. It will be understood in view of this disclosure that any suitable UV wavelengths may be used, and the disclosure is not limited in this regard. It will further be understood in view of this disclosure that suitable UV wavelengths may be selected and affected based on factors such as those described herein. Suitable UV intensities may be, or include, 50 milliwatts per square centimeter (mW/cm²) to 4000 mW/cm² or 200 mW/cm² to 1000 mW/cm². In some embodiments, the UV intensity is 500 mW/cm². It will be understood in view of this disclosure that any suitable UV intensities may be used, and the disclosure is not limited in this regard. It will further be understood in view of this disclosure that suitable UV intensities may be selected and affected based on factors such as those described herein.

As described herein, the feedthrough potting adhesive may have any suitable water uptake. Water uptake may be determined (e.g., measured) using any suitable technique. Suitable techniques for determining water uptake may include, for example, ASTM D570-98. Suitable water uptake may be selected based on, for example, maintaining electrical insulation between a pin (e.g., the pin 202) and a ferrule (e.g., the ferrule 204) of an implantable medical device (e.g., the implantable medical device 100). Water uptake may be affected by factors such as the polarity of compounds (where non-polarity tend to reduce or minimize water uptake) and hydrophobicity (as determined, e.g., by octanol-water-partition coefficient, where higher coefficient and higher hydrophobicity tend to reduce or minimize water uptake), as just two examples. In some embodiments, the feedthrough potting adhesive may have a water uptake of 5 wt-% or less, 4 wt-% or less, 3 wt-% or less, 2 wt-% or less, or 1.5 wt-% or less after 60 days in a 37° C. aqueous environment. In some embodiments, the feedthrough potting adhesive is substantially free of water.

The feedthrough potting adhesive may have any suitable glass transition temperature. Glass transition temperature may be determined (e.g., measured) using any suitable technique. Suitable techniques for determining glass transition temperature may include, for example, differential scanning calorimetry (DSC) or ASTM D5026 using dynamic mechanical analyzer (DMA). Suitable glass transition temperatures may be selected based on factors such as the ambient temperature that the feedthrough potting adhesive will be subject to (e.g., normal body temperature in the case of implantable medical devices), as an example. Suitable glass transition temperatures may include, for example, 37° C., 20° C. to 50° C., or 27° C. to 47° C. Further examples of suitable glass transition temperatures may include 55° C. or less, 50° C. or less, 40° C. or less, 37° C. or less, or 30° C. or less, and/or 25° C. or greater, 30° C. or greater, 37° C. or greater, 40° C. or greater, or 50° C. or greater.

The feedthrough potting adhesive may have any suitable impedance. Impedance may be determined (e.g., measured) using any suitable technique. Suitable techniques for determining impedance may include, for example, electrochemical impedance spectroscopy (EIS). Generally, a higher impedance is more desirable than a lesser impedance, for example, because a higher impedance (e.g., 1 gigaohm or greater at 0.1 Hz) tends to provide more effective insulation (e.g., between the ferrule 204 and the pin 202 of the feedthrough assembly 200. Suitable impedances at 0.1 Hertz (Hz) may include, for example, 1 gigaohm or greater, 3 gigaohms or greater, 10 gigaohms or greater, 30 gigaohms or greater, 100 gigaohms or greater, or 300 gigaohms or greater. Feedthrough potting adhesives according to the present disclosure have been found by the inventors to advantageously sustain high impedance (e.g., 1 gigaohm or greater at 0.1 Hz) in (e.g., during, after, etc.) aqueous environment, such as after soaking in 37° C. phosphate buffered saline (PBS). In some embodiments, the feedthrough potting adhesive has an impedance at 0.1 Hz of 1 gigaohm or greater, 3 gigaohms or greater, 10 gigaohms or greater, 30 gigaohms or greater, 100 gigaohms or greater, or 300 gigaohms or greater after 50 days or greater, 60 days or greater, 100 days or greater, 150 days or greater, 200 days or greater, 300 days or greater, or 1 year or greater in a 37° C. aqueous environment.

The feedthrough potting adhesive may have any suitable dielectric constant. Dielectric constant may be determined (e.g., measured) using any suitable technique. Suitable techniques for determining dielectric constant may include, for example, ASTM D150 using electrochemical impedance spectroscopy (EIS). Suitable dielectric constants at 37° C. may include, for example, 1.5 to 6.5 or 2 to 4.5. In some embodiments, the feedthrough potting adhesive has a dielectric constant of 2.5. In one or more embodiments, the feedthrough potting adhesive has a dielectric constant of 2.9.

The feedthrough potting adhesive may have any suitable storage modulus and/or loss modulus. A suitable storage modulus of the feedthrough potting adhesive may be selected based on factors such as desired stiffness and resistance to deformation of the feedthrough potting adhesive (i.e., the cured photocurable liquid feedthrough potting adhesive), for example. A suitable loss modulus of the feedthrough potting adhesive may be selected based on factors such as desired ability to dissipate stress, such as stress on the pin during manufacture (e.g., assembly) of the implantable medical device. A suitable storage modulus of the feedthrough potting adhesive and a suitable loss modulus of the feedthrough potting adhesive may each be selected, for example, to afford desired mechanical support to prevent a pin (e.g., the pin 202) from being deformed under stress (e.g., stress from manipulation during device assembly), and particularly to prevent to contact between a ferrule (e.g., the ferrule 204) and a pin (e.g., the pin 202).

A suitable storage modulus of the feedthrough potting adhesive may be, for example, 600 to 2000 megapascals (MPa) or 900 to 1500 MPa. As further examples, a suitable storage modulus of the feedthrough potting adhesive may be, or include, 500 MPa or greater, 600 MPa or greater, 900 MPa or greater, 1000 MPa or greater, 1500 MPa or greater, or 2000 MPa or greater, and/or 2200 MPa or less, 2000 MPa or less, 1500 MPa or less, 1000 MPa or less, 900 MPa or less, or 600 MPa or less. It will be understood in view of this disclosure that any suitable feedthrough potting adhesive storage moduli may be used, and the disclosure is not limited in this regard. It will further be understood in view of this disclosure that suitable feedthrough potting adhesive storage moduli may be selected and affected based on factors such as those described herein.

A suitable loss modulus of the feedthrough potting adhesive may be, for example 50 to 300 MPa or 70 to 200 MPa. As further example, a suitable loss modulus of the feedthrough potting adhesive may be, or include, 40 MPa or greater, 50 MPa or greater, 70 MPa or greater, 100 MPa or greater, 200 MPa or greater, or 300 MPa or greater, and/or 350 MPa or less, 300 MPa or less, 200 MPa or less, 100 MPa or less, 70 MPa or less, or 50 MPa or less. It will be understood in view of this disclosure that any suitable feedthrough potting adhesive loss moduli may be used, and the disclosure is not limited in this regard. It will further be understood in view of this disclosure that suitable feedthrough potting adhesive loss moduli may be selected and affected based on factors such as those described herein.

In one or more embodiments, as described herein, the feedthrough potting adhesive includes a polyether polymer, which is formed by reacting ingredients including a prepolymer composition, a photoinitiator, a cross-linking agent, and a hydrophobic polyol. In other words, the polyether polymer may be described as the reaction product of reaction materials including the prepolymer composition, the photoinitiator, the cross-linking agent, and the hydrophobic polyol.

The reaction materials may be selected to form any suitable polyether polymer or any suitable combination of suitable polyether polymers. Suitable polyether polymers may be substantially free or essential free of hydrolysable functionalities, for example. Polyether polymers substantially free or essentially free of hydrolysable functionalities may advantageously have improved hydrolytic stability, for example, in comparison with polymers that are not substantially free or essential free of hydrolysable functionalities. As another example, suitable polyether polymers may be substantially free or essentially free of unsaturated bonds (e.g., C═C or C═O). The polyether polymer being substantially free or essentially free of unsaturated bonds may advantageously afford resistance to oxidated degradation of the polyether polymer.

The feedthrough potting adhesive may include any suitable amount of the polyether polymer. Suitable amounts of the polyether polymer may be selected based on factors such as desired properties of the feedthrough potting adhesive (e.g., storage/loss modulus, impedance, dielectric constant, etc.). For example, the feedthrough potting adhesive may include 75 wt-% to 99 wt-% or 85 wt-% to 97 wt-% of polyether polymer based on the total weight of the feedthrough potting adhesive. As further examples, the feedthrough potting adhesive may include 70 wt-% or greater, 75 wt-% or greater, 80 wt-% or greater, 85 wt-% or greater, 90 wt-% or greater, 95 wt-% or greater, 97 wt-% or greater, 98 wt-% or greater, or 99 wt-% or greater, and/or 99.9 wt-% or less, 99 wt-% or less, 98 wt-% or less, 97 wt-% or less, 95 wt-% or less, 90 wt-% or less, 85 wt-% or less, 80 wt-% or less, or 75 wt-% or less. In some embodiments, the feedthrough potting adhesive may include 95% or greater of polyether polymer based on the total weight of the feedthrough potting adhesive.

The polyether polymer may be formed by reacting any suitable amount of the combination (e.g., mixture) of ingredients described herein (i.e., any suitable amount of prepolymer composition, photoinitiator, cross-linking agent, and hydrophobic polyol) based on the total weight of the polyether polymer or based on the total weight of the feedthrough potting adhesive. For example, 85 wt-% or greater, 90 wt-% or greater, 95 wt-% or greater, or 98 wt-% or greater of the polyether polymer may be formed by reacting the combination of prepolymer composition, photoinitiator, cross-linking agent, and hydrophobic polyol.

The polyether polymer of the feedthrough potting adhesive may have any suitable storage modulus and/or loss modulus. Suitable storage modulus of the polyether polymer may be selected based on factors such as desired stiffness and resistance to deformation of the feedthrough potting adhesive, for example. Suitable loss modulus of the polyether polymer may be selected based on factors such as desired ability of the feedthrough potting adhesive to dissipate stress. Suitable storage modulus of the polyether polymer and suitable loss modulus of the polyether polymer may each be selected, for example, to afford desired mechanical support to prevent a pin (e.g., the pin 202) from being deformed under stress (e.g., stress from manipulation during device assembly) to contact a ferrule (e.g., the ferrule 204).

A suitable storage modulus of the polyether polymer may be, for example, 600 to 2000 megapascals (MPa) or 900 to 1500 MPa. As further examples, a suitable storage modulus of the polyether polymer may be, or include, 500 MPa or greater, 600 MPa or greater, 900 MPa or greater, 1000 MPa or greater, 1500 MPa or greater, or 2000 MPa or greater, and/or 2200 MPa or less, 2000 MPa or less, 1500 MPa or less, 1000 MPa or less, 900 MPa or less, or 600 MPa or less. It will be understood in view of this disclosure that any suitable polyether polymer storage moduli may be used, and the disclosure is not limited in this regard. It will further be understood in view of this disclosure that suitable polyether polymer storage moduli may be selected and affected based on factors such as those described herein.

A suitable loss modulus of the polyether polymer may be, for example 50 to 300 MPa or 70 to 200 MPa. As further example, a suitable loss modulus of the polyether polymer may be, or include, 40 MPa or greater, 50 MPa or greater, 70 MPa or greater, 100 MPa or greater, 200 MPa or greater, or 300 MPa or greater, and/or 350 MPa or less, 300 MPa or less, 200 MPa or less, 100 MPa or less, 70 MPa or less, or 50 MPa or less. It will be understood in view of this disclosure that any suitable polyether polymer loss moduli may be used, and the disclosure is not limited in this regard. It will further be understood in view of this disclosure that suitable polyether polymer loss moduli may be selected and affected based on factors such as those described herein.

### Illustrative Aspects

Aspect 1 is an implantable medical device feedthrough assembly comprising:
a feedthrough ferrule; and
a feedthrough potting adhesive in the feedthrough ferrule, the feedthrough potting adhesive comprising a polyether polymer formed by reacting ingredients including: a photoinitiator; a hydrophobic polyol; a cross-linking agent; and a prepolymer compound including segments of the following formula: X₁—R—X₂,
wherein X₁ is an epoxide, a glycidyl ether, an oxetane, or a photoinitiated ring-opening cyclic ether; wherein X₂ is an epoxide, a glycidyl ether, an oxetane, a photoinitiated ring-opening cyclic ether, or a hydrogen; and wherein R is an organic group.

Aspect 2 is a method for forming an implantable medical device, the method comprising: bonding a feedthrough pin within a ferrule with a feedthrough potting adhesive, the feedthrough potting adhesive comprising a polyether polymer formed by reacting ingredients including: a photoinitiator; a hydrophobic polyol; a cross-linking agent; and a prepolymer compound including segments of the following formula: X₁—R—X₂,
wherein X₁ is an epoxide, a glycidyl ether, an oxetane, or a photoinitiated ring-opening cyclic ether; wherein X₂ is an epoxide, a glycidyl ether, an oxetane, a photoinitiated ring-opening cyclic ether, or a hydrogen; and wherein R is an organic group; and curing the feedthrough potting adhesive.

Aspect 3 is the assembly according to aspect 1 or the method according to aspect 2, wherein the prepolymer compound is a diepoxide compound of the following formula:

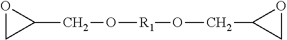

wherein R₁ is an organic group, an alkyl group, an alkyl ether group, a cycloalkane group, or an aryl group.

Aspect 4 is the assembly according to aspect 1 or the method according to aspect 2, wherein X₁, X₂, or both are an epoxide, a glycidyl ether, an oxetane, or any combination thereof, and wherein R is O—R₁—O, wherein R₁ is an organic group, an alkyl group, an alkyl ether group, a cycloalkane group, or an aryl group.

Aspect 5 is the assembly according to aspect 1 or the method according to aspect 2, wherein the prepolymer compound is a dioxetane compound of the following formula:

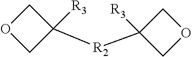

wherein each of R₂ and R₃ independently is an organic group, an alkyl group, an alkyl ether group, a cycloalkane group, or an aryl group.

Aspect 6 is the assembly according to aspect 1 or the method according to aspect 2, wherein X₁, X₂, or both are an epoxide, a glycidyl ether, an oxetane, or any combination thereof.

Aspect 7 is the assembly or the method according to any one of aspects 1-6, wherein the photoinitiator comprises a cation from an aromatic diazonium salt, an aromatic sulfonium salt, an aromatic iodonium salt, a metallocene-based compound, an aromatic phosphonium salt, or any combination thereof, and an anion from BF₄⁻, AsF₆⁻, SbF₆⁻, PF₆⁻, [B(CF₃)₄]⁻, B(C₆F₅)₄⁻, B[C₆H₃-3,5(CF₃)₂]₄⁻, B(C₆H₄CF₃)₄⁻, B(C₆H₃F₂)₄⁻, B[C₆F₄-4(CF₃)]₄⁻, or any combination thereof.

Aspect 8 is the assembly or the method according to any one of aspects 1-7, wherein the photoinitiator comprises a cation from a triarylsulfonium salt and an anion from hexafluoroantimonate.

Aspect 9 is the assembly or the method according to any one of aspects 1-8, wherein the hydrophobic polyol comprises a polyether polyol, a polyester polyol, a dimer-diol, a trimer-triol, a hydroxyl-terminated hydrogenated polybutadiene, a hydrogenated hydroxyl-terminated polyfarnesene, or any combination thereof.

Aspect 10 is the assembly or the method according to any one of aspects 1-9, wherein the cross-linking agent comprises glycidyl 3-(trimethoxysilyl)propyl ether.

Aspect 11 is the assembly or the method according to any one of aspects 1-10, wherein the feedthrough potting adhesive has a glass transition temperature of 37° C. or greater, 40° C. or greater, or 50° C. or greater.

Aspect 12 is the assembly or the method according to any one of aspects 1-11, wherein the feedthrough potting adhesive is substantially free of water.

Aspect 13 is the assembly or the method according to any one of aspects 1-12, wherein the feedthrough potting adhesive has a water uptake of 5 wt-% or less, 4 wt-% or less, 3 wt-% or less, 2 wt-% or less, or 1.5 wt-% or less after 60 days in a 37° C. aqueous environment.

Aspect 14 is the assembly or the method according to any one of aspects 1-13, wherein the feedthrough potting adhesive is curable under exposure to UV radiation in 60 minutes or less, 30 minutes or less, 15 minutes or less, 5 minutes or less, 2 minutes or less, 1.5 minutes or less, 1 minute or less, 30 seconds or less, or 15 seconds or less.

Aspect 15 is the assembly or the method according to any one of aspects 1-14, wherein the prepolymer compound is non-polar, the hydrophobic polyol is non-polar, the cross-linking agent is non-polar, or any combination thereof.

Aspect 16 is the assembly or the method according to any one of aspects 1-15, wherein the prepolymer has an octanol-water partition coefficient (Log P) of 2 or greater, the hydrophobic polyol has a Log P of 2 or greater, the cross-linking agent has a Log P of 2 or greater, the photoinitiator has a Log P of 2 or greater, or any combination thereof.

Aspect 17 is the assembly or the method according to any one of aspects 1-16, wherein the polyether polymer is substantially free or essentially free of hydrolysable functionalities, and optionally wherein the polyether polymer is substantially free or essentially free of unsaturated bonds.

Aspect 18 is the assembly or the method according to any one of aspects 1-17, wherein the feedthrough potting adhesive has an impedance at 0.1 Hertz (Hz) of 1 gigaohm or greater, 10 gigaohms or greater, or 100 gigaohms or greater.

Aspect 19 is the assembly or the method according to aspect 18, wherein the feedthrough potting adhesive has the impedance at 0.1 Hz after 50 days or greater, 60 days or greater, 100 days or greater, 150 days or greater, 200 days or greater, 300 days or greater, or 1 year or greater in a 37° C. aqueous environment.

Aspect 20 is the assembly or the method according to any one of aspects 1-19, wherein the feedthrough potting adhesive has a storage modulus of 600 to 2000 megapascals (MPa) or 900 to 1500 MPa.

Aspect 21 is the assembly or the method according to any one of aspects 1-20, wherein the feedthrough potting adhesive has a loss modulus of 50 to 300 MPa or 70 to 200 MPa.

Aspect 22 is the assembly or the method according to any one of aspects 1-21, wherein a combination of the ingredients has a thixotropic index of 1.0 to 1.2.

Aspect 23 is the assembly or the method according to any one of aspects 1-22, wherein a combination of the ingredients has a viscosity of 2500 centipoise (cps) or less, 2000 cps or less, 1500 cps or less, 1000 cps or less, 500 cps or less, or 300 cps or less.

Aspect 24 is the assembly or the method according to aspect 23, wherein the combination of the ingredients has the viscosity after 2 hours or greater at an ambient temperature of 23° C.

Aspect 25 is the method according to any one of aspects 1-24, wherein the curing the feedthrough potting adhesive occurs in 60 minutes or less, 30 minutes or less, 15 minutes or less, 5 minutes or less, 2 minutes or less, or 1.5 minutes or less.

Aspect 26 is a feedthrough potting adhesive comprising: a polyether polymer formed by reacting ingredients including: a photoinitiator; a hydrophobic polyol; a cross-linking agent; and a prepolymer compound including segments of the following formula: X₁—R—X₂, wherein X₁ is an epoxide, a glycidyl ether, an oxetane, or a photoinitiated ring-opening cyclic ether; wherein X₂ is an epoxide, a glycidyl ether, an oxetane, a photoinitiated ring-opening cyclic ether, or a hydrogen; and wherein R is an organic group.

### Examples

TABLE 1
Materials used in the Examples.

| Material ID | Description | Source |
|---|---|---|
| DGEBPA | Diglycidyl ether of bisphenol A resin, | WESTLAKE EPOXY |
| | commercially available as EPON RESIN | INC., Stafford, Texas, |
| | 828. | USA |
| BGE | Butyl glycidyl ether resin, commercially | WESTLAKE EPOXY |
| | available as HELOXY MODIFIER 61. | INC., Stafford, Texas, |
| | | USA |
| BDDGE | 1,4-Butanediol diglycidyl ether, | WESTLAKE EPOXY |
| | commercially available as HELOXY | INC., Stafford, Texas, |
| | MODIFIER 67. | USA |
| TCDDGE | Tricyclodecane dimethanol diglycidylether, | ADEKA |
| | commercially available as EP4088S. | CORPORATION, |
| | | Tokyo, Japan. |
| HBPADGE | Hydrogenated bisphenol A type epoxy | ADEKA |
| | resin, commercially available as EP- | CORPORATION, |
| | 4080E. | Tokyo, Japan. |
| DGEBPA-PO | Propoxylated Diglycidyl ether of bisphenol | ADEKA |
| | A resin, commercially available as EP- | CORPORATION, |
| | 4000S. | Tokyo, Japan. |
| GLYMO | Glycidyl 3-(trimethoxysilyl)propyl ether, | EVONIK |
| | commercially available as DYNASYLAN | CORPORATION, |
| | GLYMO. | Parsippany, NJ, USA |
| DOX | 3,3′-(Oxybis(methylene))bis(3- | PERSTORP |
| | ethyloxetane), commercially available as | SPECIALTY |
| | CURALITE OXPLUS. | CHEMICALS AB, |
| | | Perstorp, Sweden |
| EOXA | 3-Ethyl-3-hydroxymetyloxetane, | PERSTORP |
| | commercially available as CURALITE | SPECIALTY |
| | OX. | CHEMICALS AB, |
| | | Perstorp, Sweden |
| P1075 | Dimer diamine, commercially available as | CRODA INC., |
| | PRIAMINE 1075. | Plainsboro, NJ, USA |
| P2043 | Dimer diol, commercially available as | CRODA INC., |
| | PRIPOL 2043. | Plainsboro, NJ, USA |
| DETA | Diethylene triamine hardener, | BASF, Florham Park, |
| | commercially available as DETA. | NJ, USA |
| SE21 | 3,4-Epoxycyclohexylmethyl-3,4- | SYNASIA INC., |
| | epoxycyclohexanecarboxylate, | Metuchen, NJ, USA |
| | commercially available as SYNA EPOXY | |
| | 21. | |
| PI | Photoinitiator from triarylsulfonium | SYNASIA INC., |
| | hexafluoroantimonate salts, commercially | Metuchen, NJ, USA |
| | available as UVI-6976. | |
| FS | Fumed silica, commercially available as | CABOT |
| | CAB-O-SIL TS-610. | CORPORATION, |
| | | Billerica, MA, USA |

The properties of a curable liquid feedthrough potting adhesives and feedthrough potting adhesives formed therefrom were tested. Photocurable liquid feedthrough potting adhesives and feedthrough potting adhesives formed therefrom (Samples EX1, EX2, EX3, and EX4) were prepared according to aspects of the present disclosure (using, e.g., prepolymer compounds, photoinitiators, cross-linking agents, hydrophobic polyols, etc. as described herein). A thermally-curable liquid feedthrough potting adhesive and a feedthrough potting adhesive formed therefrom (Sample CE) was also prepared.

TABLE 2
Curable liquid feedthrough potting adhesive compositions.

Concentration (wt-%)

| Material ID | CE | EX1 | EX2 | EX3 | EX4 |
|---|---|---|---|---|---|
| DGEBPA | 55.5 | — | — | 20.0 | — |
| DGEBPA-PO | — | — | — | — | 30 |
| BGE | 6.9 | — | — | — | — |
| BDDGE | — | — | — | 26.5 | — |
| TCDDGE | — | — | 45.0 | — | — |
| HBPADGE | — | 45.0 | — | — | — |
| GLYMO | — | 1.5 | 1.5 | 1.0 | 3.0 |
| DOX | — | 27.0 | 30.0 | — | 23.5 |
| EOXA | — | — | — | — | 32.0 |
| P1075 | 35.7 | — | — | — | — |
| P2043 | — | 25.0 | 22.0 | — | — |
| DETA | 1.9 | — | — | — | — |
| SE21 | — | — | — | 51.0 | — |
| PI | — | 1.5 | 1.5 | 1.5 | 1.5 |
| FS | — | — | — | — | 10.0 |

### Sample Preparation

Samples CE, EX1, EX2, EX3, and EX4 were prepared using the materials and amounts shown in Table 2. The materials were weighed, mixed, and then degassed under 50 millibars (mbar) vacuum. The resulting curable liquid feedthrough potting adhesive of each sample was tested for pot life and thixotropic index. The curable liquid feedthrough potting adhesives of each sample was cured to form a cured feedthrough potting adhesive. The cured feedthrough potting adhesive of each sample was tested for water uptake, glass transition temperature, storage modulus, loss modulus, impedance, and current leakage.

### Pot Life

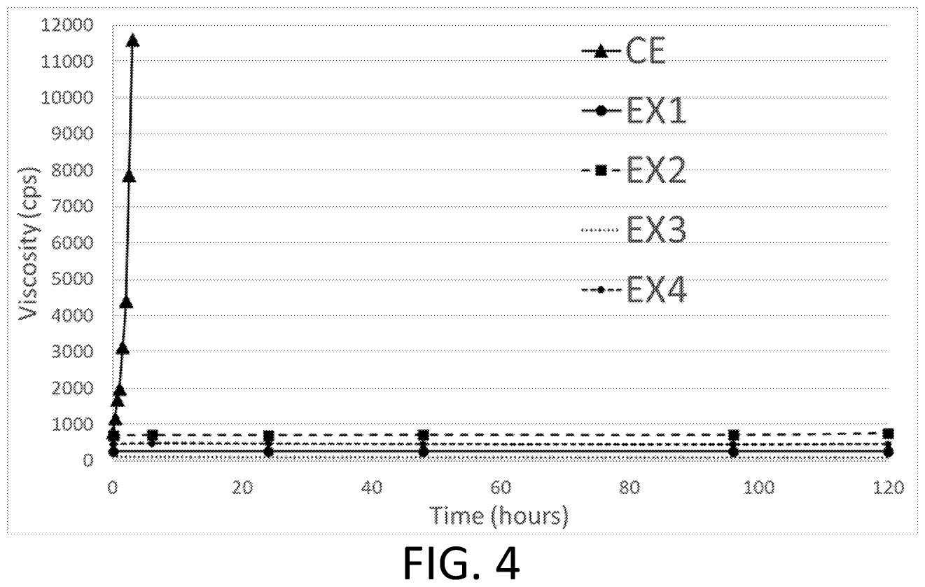

The pot life of the curable liquid feedthrough potting adhesive of each sample was determined as the time to reach a viscosity of 2,500 cps or greater at an ambient temperature of 23° C. Viscosity was measured at a shear rate of 10/s, according to ASTM D1084. The results are shown in FIG. 4. The viscosity of EX1 after 48 hours at 23° C. was 257 cps, (unchanged from the initially measured viscosity). The viscosity of EX2 after 48 hours at 23° C. was 753 cps (increased from the initially measured viscosity of 691 cps). The viscosity of EX3 after 48 hours at 23° C. was 104 cps (unchanged from the initially measured viscosity). The viscosity of EX4 after 48 hours at 23° C. was 458 (unchanged from the initially measured viscosity). The pot life of CE was less than 2 hours at an ambient temperature of 23° C. The viscosity of CE after 2 hours at 23° C. was 4,390 cps, (a 556% increase from the initially measured viscosity of 789 cps).

### Thixotropic Index

The thixotropic index of the curable liquid feedthrough potting adhesive of each sample was determined as the ratio of the viscosity at 1/s shear rate over the viscosity at 10/s shear rate. As described herein, a low thixotropic index of 1.0 to 1.2 may be desirable for feedthrough potting applications, for example, because a low thixotropic index of 1.0 to 1.2 may be described as characteristic of a liquid with free flowing and self-leveling properties. The measured thixotropic index of each of CE, EX1, EX2, EX3, and EX4 is 1.0.

### Water Uptake

Water uptake of the cured feedthrough potting adhesive of each sample was measured after each cured sample had soaked in 37° C. deionized (DI) water for 60 days. The water uptake was measured in accordance with ASTM D570-98. The results are shown in Table 3. Low water uptake of (e.g., 2.0 wt-% or less) may be desirable in feedthrough potting adhesive applications, for example, for maintenance of adhesion, such as between the cured feedthrough potting adhesive and a feedthrough ferrule (e.g., the ferrule 204). CE, EX1, and EX2 each have very low water uptake of 1.5%, 1.2%, and 1.3%, respectively. EX3 has 6.0% water uptake. Without wishing to be bound by theory, because EX3 contains no hydrophobic chain transfer cross-linker P2043, it was observed that the hydrophobic chain transfer cross-linkers may afford a cured feedthrough potting adhesive with improved water repellency.

TABLE 3
Water uptake of cured feedthrough potting adhesive
samples after 60-day DI water soak at 37° C.

| | CE | EX1 | EX2 | EX3 | EX4 |
|---|---|---|---|---|---|
| Water uptake (wt-%) | 1.5 | 1.2 | 1.3 | 6.0 | 2.1 |

### Glass Transition Temperature

The glass transition temperature (Tg) of the cured feedthrough potting adhesive of each sample was determined by DMA, according to ASTM D7028. The results are shown in Table 4. It may be desirable for the Tg of the cured feedthrough potting adhesive to be greater than 37° C. (i.e., human body temperature), for example, for improved stability in implantable medical device applications. The Tg of each sample was observed to be greater than 37° C.

TABLE 4
Glass transition temperature of cured
feedthrough potting adhesive samples.

| | CE | EX1 | EX2 | EX3 | EX4 |
|---|---|---|---|---|---|
| Tg (° C) | 52 | 53 | 51 | 51 | 45 |

### Storage Modulus and Loss Modulus

The storage modulus and loss modulus at 37° C. of the cured feedthrough potting adhesive of each sample was determined by DMA, according to ASTM D5026. The results are shown in Table 5. As described herein, the storage modulus may be described as indicative of a material's stiffness and resistance to deformation and loss modulus may be described as indicative of a material's ability to dissipate stress. Generally, the storage modulus of a cured feedthrough potting adhesive may preferably be 900 MPa to 1500 MPa. The determined storage modulus of each cured sample was observed to be within the preferred range of 900 MPa to 1500 MPa. Generally, the loss modulus of a cured feedthrough potting adhesive may preferably be 70 MPa to 200 MPa. The determined loss modulus of each cured sample was observed to be within the preferred range of 70 MPa to 200 MPa.

TABLE 5
Storage and loss modulus of cured feedthrough
potting adhesive samples.

| | CE | EX1 | EX2 | EX3 | EX4 |
|---|---|---|---|---|---|
| Storage Modulus (MPa) | 1138 | 1136 | 1040 | 1175 | 1133 |
| Loss Modulus (MPa) | 103 | 90 | 83 | 167 | 163 |

### Impedance

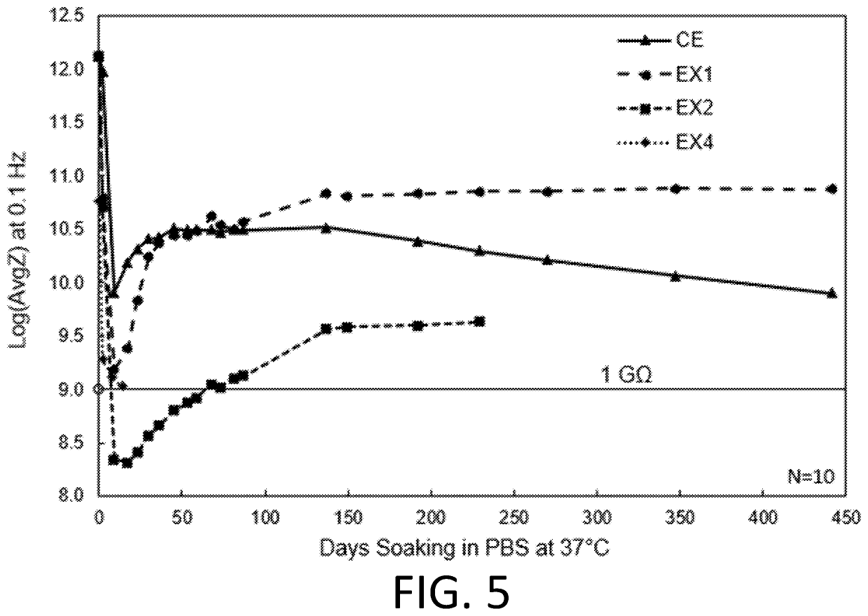

The impedance of the cured feedthrough potting adhesive of each of samples CE, EX1, EX2, and EX4 was determined using electrochemical impedance spectroscopy (EIS). Each sample was soaked in phosphate buffered saline (PBS) at 37° C. and the impedance of each sample was periodically measured. The results are shown in FIG. 5. In comparison to sample CE, sample EX1 was observed to have similarly high impedance and stability over a test period of 100 days. In further comparison to sample CE, sample EX1 was observed to have greater impedance and stability over a test period of 450 days.

### Current Leakage

The current leakage of the cured feedthrough potting adhesive of each of samples CE and EX1 was determined using high voltage shock testing. Respective feedthrough assemblies were prepared, each having 10 ferrules with a pin bonded therein using the cured feedthrough potting adhesive of one of samples CE or EX1. For each feedthrough assembly, high-voltage (HV) pulses were applied to all 10 feedthroughs, which were connected in parallel. The pulses were 1,500 volt monophasic waveforms with 50% tilt, 18 ms (millisecond) pulse width and 1 second intervals. Each feedthrough assembly was soaked in PBS buffer at 37° C. and was periodically characterized by HV testing. Each feedthrough assembly received a total of 161 HV pulses over a period of 12 weeks. For each feedthrough assembly, the maximum current across all feedthroughs for any single pulse was recorded and tracked versus time. The results are shown in Table 6. Both sample CE and sample EX1 were observed to have low current leakage of close to zero.

TABLE 6
Currently leakage of cured feedthrough potting adhesive.

| | |
|---|---|
| High Voltage Shock Test | Current Leakage (milliAmps) |

| Days soaked in 37° C. PBS | CE | EX1 |
|---|---|---|
| 2 | 0.082 | 0.075 |
| 9 | 0.081 | 0.339 |
| 17 | 0.066 | 0.203 |
| 23 | 0.069 | 0.159 |
| 30 | 0.071 | 0.133 |
| 36 | 0.059 | 0.112 |
| 45 | 0.061 | 0.088 |
| 53 | 0.079 | 0.087 |
| 59 | 0.058 | 0.070 |
| 68 | 0.058 | 0.077 |
| 73 | 0.071 | 0.072 |
| 81 | 0.092 | 0.093 |

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An implantable medical device feedthrough assembly comprising:
a feedthrough ferrule; and
a feedthrough potting adhesive in the feedthrough ferrule, the feedthrough potting adhesive comprising a polyether polymer formed by reacting ingredients including:
a photoinitiator;
a hydrophobic polyol;
a cross-linking agent; and
a prepolymer compound including segments of the following formula: 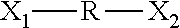
wherein X₁ is an epoxide, a glycidyl ether, an oxetane, or a photoinitiated ring-opening cyclic ether;
wherein X₂ is an epoxide, a glycidyl ether, an oxetane, a photoinitiated ring-opening cyclic ether, or a hydrogen; and
wherein R is an organic group.

2. The assembly according to claim 1, wherein the prepolymer compound is a diepoxide compound of the following formula: 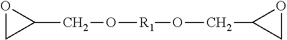
wherein R₁ is an organic group, an alkyl group, an alkyl ether group, a cycloalkane group, or an aryl group.

3. The assembly according to claim 1, wherein X₁, X₂, or both are an epoxide, a glycidyl ether, an oxetane, or any combination thereof, and wherein R is O—R₁—O, wherein R₁ is an organic group, an alkyl group, an alkyl ether group, a cycloalkane group, or an aryl group.

4. The assembly according to claim 1, wherein the prepolymer compound is a dioxetane compound of the following formula: 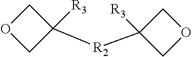
wherein each of R₂ and R₃ independently is an organic group, an alkyl group, an alkyl ether group, a cycloalkane group, or an aryl group.

5. The assembly according to claim 1, wherein the photoinitiator comprises a cation from an aromatic diazonium salt, an aromatic sulfonium salt, an aromatic iodonium salt, a metallocene-based compound, an aromatic phosphonium salt, or any combination thereof, and an anion from BF₄⁻, AsF₆⁻, SbF₆⁻, PF₆⁻, [B(CF₃)₄]⁻, B(C₆F₅)₄⁻, B[C₆H₃-3,5(CF₃)₂]₄⁻, B(C₆H₄CF₃)₄⁻, B(C₆H₃F₂)₄⁻, B[C₆F₄-4(CF₃)]₄⁻, or any combination thereof.

6. The assembly according to claim 1, wherein the photoinitiator comprises a cation from a triarylsulfonium salt and an anion from hexafluoroantimonate.

7. The assembly according to claim 1, wherein the hydrophobic polyol comprises a polyether polyol, a polyester polyol, a dimer-diol, a trimer-triol, a hydroxyl-terminated hydrogenated polybutadiene, a hydrogenated hydroxyl-terminated polyfarnesene, or any combination thereof.

8. The assembly according to claim 1, wherein the cross-linking agent comprises glycidyl 3-(trimethoxysilyl)propyl ether.

9. The assembly according to claim 1, wherein the feedthrough potting adhesive has a glass transition temperature of 37° C. or greater, 40° C. or greater, or 50° C. or greater.

10. The assembly according to claim 1, wherein the feedthrough potting adhesive has a water uptake of 5 wt-% or less after 60 days in a 37° C. aqueous environment.

11. The assembly according to claim 1, wherein the prepolymer compound is non-polar, the hydrophobic polyol is non-polar, the cross-linking agent is non-polar, or any combination thereof.

12. The assembly according to claim 1, wherein the prepolymer has an octanol-water partition coefficient (Log P) of 2 or greater, the hydrophobic polyol has a Log P of 2 or greater, the cross-linking agent has a Log P of 2 or greater, the photoinitiator has a Log P of 2 or greater, or any combination thereof.

13. The assembly according to claim 1, wherein the polyether polymer is substantially free or essentially free of hydrolysable functionalities, and optionally wherein the polyether polymer is substantially free or essentially free of unsaturated bonds.

14. The assembly according to claim 1, wherein the feedthrough potting adhesive has the impedance at 0.1 Hz after at least 1 year in a 37° C. aqueous environment.

15. The assembly according to claim 1, wherein the feedthrough potting adhesive has a loss modulus of 50 MPa to 300 MPa.

16. The assembly according to claim 1, wherein a combination of the ingredients has a thixotropic index of 1.0 to 1.2.

17. The assembly according to claim 1, wherein a combination of the ingredients has a viscosity of 2500 centipoise (cps) or less after 2 hours or greater at an ambient temperature of 23° C.

18. A feedthrough potting adhesive comprising:
a polyether polymer formed by reacting ingredients including:
a photoinitiator;
a hydrophobic polyol;
a cross-linking agent; and
a prepolymer compound including segments of the following formula: 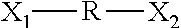
wherein X₁ is an epoxide, a glycidyl ether, an oxetane, or a photoinitiated ring-opening cyclic ether;
wherein X₂ is an epoxide, a glycidyl ether, an oxetane, a photoinitiated ring-opening cyclic ether, or a hydrogen; and
wherein R is an organic group.

19. A method for forming an implantable medical device, the method comprising:
bonding a feedthrough pin within a ferrule with a feedthrough potting adhesive, the feedthrough potting adhesive comprising a polyether polymer formed by reacting ingredients including:
a photoinitiator;
a hydrophobic polyol;
a cross-linking agent; and
a prepolymer compound including segments of the following formula: 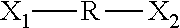
wherein X₁ is an epoxide, a glycidyl ether, an oxetane, or a photoinitiated ring-opening cyclic ether;
wherein X₂ is an epoxide, a glycidyl ether, an oxetane, a photoinitiated ring-opening cyclic ether, or a hydrogen; and
wherein R is an organic group; and
curing the feedthrough potting adhesive.

20. The method according to claim 19, wherein the curing the feedthrough potting adhesive occurs in 60 minutes or less.
